(19) United States
(12) Patent Application Publication
(10) Pub. No.: US 2016/0262922 A1
(43) Pub. Date: Sep. 15, 2016

(54) FACE FORM GUARD

(71) Applicants: Valdemar Portney, Newport Coast, CA (US); Zoya Spivak, Redondo Beach, CA (US)

(72) Inventors: Valdemar Portney, Newport Coast, CA (US); Zoya Spivak, Redondo Beach, CA (US)

(21) Appl. No.: 15/058,386

(22) Filed: Mar. 2, 2016

Related U.S. Application Data
(60) Provisional application No. 62/131,212, filed on Mar. 10, 2015.

(51) Int. Cl. A61F 5/01; A61F 5/37

(52) U.S. Cl. CPC A61F 5/01; A61F 5/3707; A61F 2005/0197

(57) ABSTRACT

A device attached to a person's head constrains movement of the face soft tissue, particularly during strenuous exercise with vigorous head movement. The device minimizes the retaining ligament's stretching and potential weakening which occurs as a natural aging process leading to a premature aging manifestation in a form of face sagging and drooping. The device includes a generally U-shaped head attachment body configured to be disposed behind the person's head and neck area and to be in contact at or near a nape area of the person. A left temporal element configured to be in contact with the person's left temporal area. A right temporal element configured to be in contact with the person's right temporal area. A left cheek supporting element configured to be in contact with the person's left cheek area. A right cheek supporting element configured to be in contact with the person's right cheek area.

### CROSS-REFERENCE TO RELATED APPLICATIONS

The present application claims priority from U.S. Provisional Patent Application Ser. No. 62/131,212 filed Mar. 10, 2015. This application is to be incorporated in its entirety into the present application with this reference.

### FIELD OF THE INVENTION

The present invention relates generally to anti-aging products. More particularly, the present invention relates to a device that constrains face soft tissue relative movement to the rest of the head particularly during strenuous exercise with vigorous head movement thus minimizing retaining ligaments stretching and potential weakening over one occurs as a natural aging process.

### BACKGROUND OF THE INVENTION

A sagging and drooping face is one of the most prominent signs of aging. The major non-behavior and non-medical factors contributing to facial aging include skeletal remodeling, skin laxity, subcutaneous fat redistribution and loss. The skin needs collagen to look younger and tighter. Once person reaches about 25 years of age, the production of collagen begins to slow down and the skin becomes drier and loose. There are many cream products to tighten the skin to some degree as well as cosmetic procedures that can temporary reverse this problem, but they're invasive and require weeks of recovery time. An example of a cosmetic procedure can be found in U.S. Pat. No. 8,974,442 by Boss, Jr., so called “a rejuvenation method” which combines skin tightening with the use of different types of energy.

There are also several anti-wrinkles devices on the market. For instance a device called The Face Wrap™ claimed to be an all-natural, non-surgical, miniface lift to tighten the skin and sculpts the face as it draws out impurities from beneath the skin. (www.thefacewrap.com/facewrap.htm) Another device is called the “Face Bra”. It is a band that slides over the top of the back of the head and then the frame fits onto the nose. (www.thegloss.com/2013/10/04/beauty/stop-aging-with-this-anti-aging-face-bra/) It was designed with certain curves to feel comfortable on certain range of face shapes. The ‘wire’ of the device intends to place a gentle pressure on the cheeks for the purpose to mitigate the smile lines that grow more prominently with age.

As people age, they lose volume in their faces, due to loss of fatty tissues. The fat pockets that benefit cheekbones and upper sides of the mouth on their faces deteriorate with age and can no longer support their face skin. The youthful face is characterized by a diffuse, balanced distribution of superficial and deep fat. With aging, in addition to redistribution of facial fat and concomitant loss of surrounding fullness, the fat pockets become more discernible as separate entities. Fat redistribution and loss is a major aging factor which is currently partially compensated by fillers through cosmetic procedures.

What does hold the fat pockets in their relative positions? It is only fairly recently been recognized that the fibrous and fat components in the subcutaneous tissue are not a uniform but arranged in discrete compartments. Over specific sites, due to the prominence of the subcutaneous fat, it has been given specific names such as the malar (related cheekbone) fat pad and nasolabial (related to nose and upper lip) fat and others. The boundary of these subcutaneous compartments corresponds to the location of the retaining ligaments, which pass superficially to insert into the dermis. In youth, transition between compartments is smooth and non discernible. With aging, as retaining ligaments weaken, skin laxity increase and other factors, a series of concavities and convexities develop which separates these compartments. Thus, the key factor is that distinct compartmentalization by the retaining ligaments holds the fat in its relative position.

The soft tissue of the face covers the underlying bony structures and is supported by ligaments that run from deep within the dermis to the overlying layers of the skin, serving as anchoring units for the face. Retaining ligaments are rigid in youth keeping the mobile superficial face tissues firmly anchored to underlying skeleton or deep fascia. Aging, together with years of muscular activity and gravity result in skin and ligamentous laxity which in combination with dermal elastosis and increased fat in deep compartment results in descent of all elements of soft tissues what is called “face sagging and drooping”.

There has been on going effort for some time to develop approaches against manifestation of aging and particularly face sagging with the focus on skin rejuvenation with the help of skin products, few devices and cosmetic procedures for skin tightening. The current application of retaining ligaments anatomical structures is also in a sight of the cosmetic surgery. The surgical correction of the retaining ligaments of the face that have descended with gravitation, are common techniques used in rejuvenation of the face, but it is expensive, not without a safety and efficacy risk, involves lengthily recovery time.

A mitigation of a tension on retaining ligaments of the face that can be significantly elevated during strenuous exercises involving sudden or significant head movement and thus contributing to face sagging has not been addressed and is the subject of the present disclosure.

### SUMMARY OF THE INVENTION

The retaining ligaments are aponeurotic condensations of fibrous tissue that run from deeper structures to overlying dermis and help to anchor the skin and mobile soft tissues to the underlying skeleton. This multilinked fibrous system attenuates with aging and gravity exacerbated by a strenuous exercise. The fat pats of the face redistribute (face sagging and drooping) with decreasing strength of the ligaments, their stretching and increasing skin laxity.

In some respect the reasons for face sagging are similar to breast sagging with age. The breasts are actually secured by connective tissue called Cooper's ligament. This connective ligament runs from the clavicle through and around breast tissue to the dermis of the skin overlying the breast. Without the internal support of the Cooper's ligament, the breast tissue sags under its own weight and loses its normal shape and contour. Breast tissue is heavier than the surrounding fat, and strain to this ligament can lead to sagging. Anything that damages the Cooper's ligament can cause the breasts to sag over time. These things include stretching or pulling on the ligament through strenuous exercise such as when the breasts bounce up and down.

Gravity pulls soft tissue down and mass inertia during excursive pulls soft tissue in different directions in sync with the direction of movement thus stretching Cooper's ligaments of the breast and retaining ligaments of the face. Due to a difference in weights of the tissues of face and breast, the breast sagging is usually more pronounced and observed earlier. Nevertheless, the direction of gravitational pull on the cheek mass, particularly for people with a higher mass and engaging in strenuous exercise, exacerbates face sagging and drooping.

A properly designed sport bra helps to prevent damaging effect on Cooper's ligament and skin stretching in strenuous exercise. The present invention discloses the face anti-sagging device to support face and mitigate stretching of the face retaining ligaments, particularly buccal maxillary ligaments as the weakest ligaments, and stretching the skin during a strenuous exercise (i.e. running, etc.). Such a device can be an excelled addition to other products that tighten the skin and together may slow down face aging manifestation.

A device according to the present invention incorporates at least two members: head attachment and form retainer. Elastic solid head attachment member is designed to be secured onto the head of different shapes and sizes as well as to manifest an inconspicuous appearance with a minimum interaction with hairstyle, head covers and eyeglasses. The form retainer member consisting of two retainer elements placed at left and right sides of the face that are fixated onto the head attachment and intent to support the cheeks for restricting their movement during head motion. The retainer elements leave a chin movement unrestricted for talking and eating.

The head attachment is in contact with the head at three areas of the head: nape of the head and both areas of the temporal regions of head that are in front of the ears tops. The nape of the head is the bottom hairline in the back of the head where the skull meets the neck. Temporal region is a region behind the eyes. The corresponding three areas of the head are practically immobile with head movement and head attachment structure lies within these three areas leading to a highly secured head attachment placement on the head.

Retainer elements may be attached to head attachment as undetachable construction or have detachable construction for replacement or position adjustment. In a latter option, left and right retainer elements are attached into the head attachment by different options, i.e. magnetic or mechanical ones. Each retaining element may be attached to the temporal element located at the same side of the head or to the head attachment body. In the latter option the retaining elements may be attached above the ear or below the ear.

In another embodiment, each right and left retainer element consists of an arm for fixation into the temporal element and cheek supporting element which contacts the cheek directly or via a disposable pad. A supporting element manifests a large enough area to comfortably supporting the cheek to largely immobilize its movement relative to the rest of the head. It may cover about half of the cheek area, such as the lower part of the cheek. It may incorporate multiple openings for light weight and air ventilation with the cheek skin area that is in contact with the supporting elements. A disposable pad might be made of a breathable and sweat absorbing material.

The described device, face form guard, may include multiple additional functions as being also a wearable device. It may include a wireless communication (phone, radio, camera, GPS, emergency signal, etc.). It may include a communication with a smart phone via Bluetooth or other technique. For instance, bone conducting technique can be used for headphone function by utilizing temple contact. The device may also include sensors for vital body measurements such as heard rate (temple contact is particularly useful for such a measure), temperature, respiration (sensors placed at supporting element may detect a sound of an air intake and outtake that passes through mouth and/or nose), oxygen saturation (skin sensors) and blood pressure (ultrasound technique, for instance). This is in addition to activity level measure by recording body movement that can be translated into the calories burned.

For better understanding of the invention, its various features and operational advantage the reference shall be made to the accompanying figures.

### BRIEF DESCRIPTION OF THE DRAWINGS

The accompanying drawings illustrate preferred embodiments of the invention. In such drawings:

FIG. 1 illustrates face of a hypothetical youthful face with estimated locations of rigid retaining ligaments. It also illustrates an approximate position of the malar fat pad as important contributor to a perception of youthful face shape.

FIG. 2 illustrates face sagging and drooping of the hypothetical aged face of the FIG. 1 with estimated putative changes in retaining ligaments that are stretched with aging, gravitational tension on them and/or exercises. It also illustrates an approximate position of the malar fat pad descend which is impacted by weakening of the retaining ligaments.

FIG. 3 demonstrates right side view of another embodiment of the device according to the present invention, called the face form guard, as being attached onto the head.

FIG. 4 demonstrates front view of the same face form guard attached to the head shown in FIG. 3.

FIG. 5 shows a close up side view of the same face form guard attached to the head shown in FIG. 3.

FIG. 6 shows a front view of the same face form guard attached to the head shown in FIG. 5.

FIG. 7 demonstrates upper view of stand along face form guard shown in FIGS. 4 and 6.

### DETAILED DESCRIPTION OF THE DRAWINGS

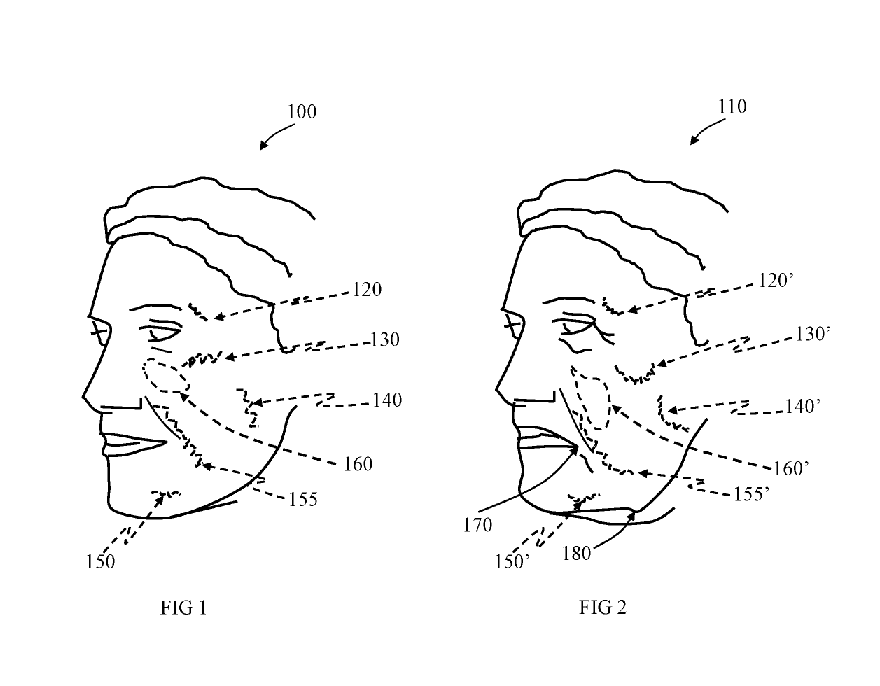

FIG. 1 illustrates a hypothetical youthful face 100 where the midface is characterized by prominent cheeks and a smooth transition between the lower eyelid and cheek supported by malar fat pad 160. A fat is also supported by rigid orbital ligaments 120. Zygomatic ligaments 130 attach the fat to the underlying zygomatic eminence and restrain the facial skin against gravitational changes and delineates the anterior border of the cheeks. Masseteric ligaments 140 support the facial fat and prevents its interior descent to the mandibular border (area at the posterior part of the lower jaw). Tissues of medial cheek are also supported by the masseteric ligaments 140. The mandibular ligaments 150 attach the parasymphyseal dermis to the underlying bone and help support the chin pad to the underlying bone. The soft tissues of the anterior cheek are supported by buccal maxillary ligaments 155 situated adjacent to the nasolabial folds.

FIG. 2 illustrates face sagging and drooping of a hypothetical aged face 110 with estimated changes from the youthful face 100 in FIG. 1. The FIG. 2 face is shown with putative changes in retaining ligaments that are stretched with aging, gravitational tension and/or exercise. Weakening of orbital ligaments 120′ effects drooping eyelids at the temporal sides. Their weakening together with weakening of zygomatic ligaments 130′ effect the malar fat pad 160′ which then descends. Laxity of masseteric ligaments 140′ leads to the characteristic submalar hollow in the aging face (a mid cheek cavity). Buccal maxillary ligaments 155′ are weakest of retaining ligaments making the anterior cheek more susceptible to sagging with aging. Their laxity elevates tension on other retaining ligaments leading to face sagging and drooping. Thus, the loss particularly of buccal maxillary ligaments 155′ support allows descent of facial fat to the mandibular border, leading to the formation of facial jowls 180. Weakening of buccal maxillary ligaments 155′ and mandibular ligaments 150′ effect nasolabial fat pad descends that together with malar fat 160′ descends lead to a pronounced “smile line” and drooping corners of mouth 170.

FIG. 3 demonstrates a side view of a human head 200 with the device according to the present invention attached to it and incorporating two members: head attachment 210 and form retainer consisting of right retainer element 220 at the right side of the head and mirror positioned and shaped left retainer element 220′ at the left side of the head.

The head attachment 210 includes head attachment body 230 as an elastic solid piece made of an elastic material and connected at one front end to the temporal element 240 that is in contact with the head at right temporal region and another temporal element 240′ symmetrically connected to the other end of the head attachment body 230 to be in contact with left temporal region of the head. Both right and left temporal elements (240, 240′) are in contact with the head at temporal area in front of the ears. Head attachment body 230 is made of elastic solid material, such as a spring metal, polymer, composite and/or any combination thereof, to accommodate a variety of human head sizes. The head attachment body 230 can be made as a single, unitary part such that it is designed to fit a majority of human heads. Alternatively, the head attachment body 230 can be made in at least two parts where a user can change the sizing such that a more custom fit can be obtained. For example, the head attachment body 230 can include a slide feature along the portion that fits behind the user's neck such that the spacing between the temporal elements 240 and 240′ can be adjusted.

A contact area of each temple element is small enough for an inconspicuous appearance, say down to about 25 mm by 25 mm minimum and large enough, say up to about 50 mm by 50 mm maximum, to minimize a pressure on the temple areas of the head. Each temporal element may include a disposable soft pad to be in contact with cheek skin and made of silicone or other material for a comfortable contact with the cheek surface.

The back side of the attachment body 230 is large enough to extend beyond most human head sizes and internally includes elastic stretchable band 250 located at the interior of the attachment body 230. This elastic stretchable band 250 is for pressing against the head at about the nape of the head. A tension of the elastic band 250 to the head is adjustable as explained below. Thus, the head attachment body 230 is secured onto the head at three head areas: the right temporal area by the right temporal element 240, the left temporal element at the left temporal area, and also at the nape area of the head.

Retainer elements (220, 220′) can be made of a solid material (spring metals, polymers, composites and/or combinations thereof) and are fixed onto the head attachment body 230 directly or via the temporal elements as shown in FIG. 3—where the right retainer 220 is attached to the right temporal element 240 and left retainer element 220′ is attached to the left temporal element 240′. The retainer elements fixation to the head attachment can be magnetic, for instance a temporal element includes a magnet and retaining element includes a magnet or is made of a ferromagnetic material that includes iron added at the retainer element end that is in contact with the temporal element. Alternatively, a fixation may rely on a mechanical one as a clamp type, for instance.

The shape of each retainer element is such that only corresponding supporting element 260 of right retainer element 220 and supporting element 260′ of left retainer element 220′ are in contact with cheeks close to the area where buccal maxillary ligaments are—i.e. in the lower half of the cheeks. Supporting elements 260, 260′ are positioned to mitigate tension on the buccal maxillary ligaments which are the weakest retaining ligaments and their weakening elevates tension on other retaining ligaments leading to an overall face sagging and drooping. Each supporting elements is approximately 15 mm of a minimum dimension and having different shapes and to about 50 mm of a maximum dimension of different shapes. Thus, the supporting elements by restricting cheeks movement relative to the rest of the head reduces tension on the retaining ligaments and particularly on the buccal maxillary ligaments. This then helps to minimize retaining ligaments stretching particularly during a strenuous exercise when head movement leads to tension elevation on the retaining ligaments by the face/cheeks soft tissue mass relative movement.

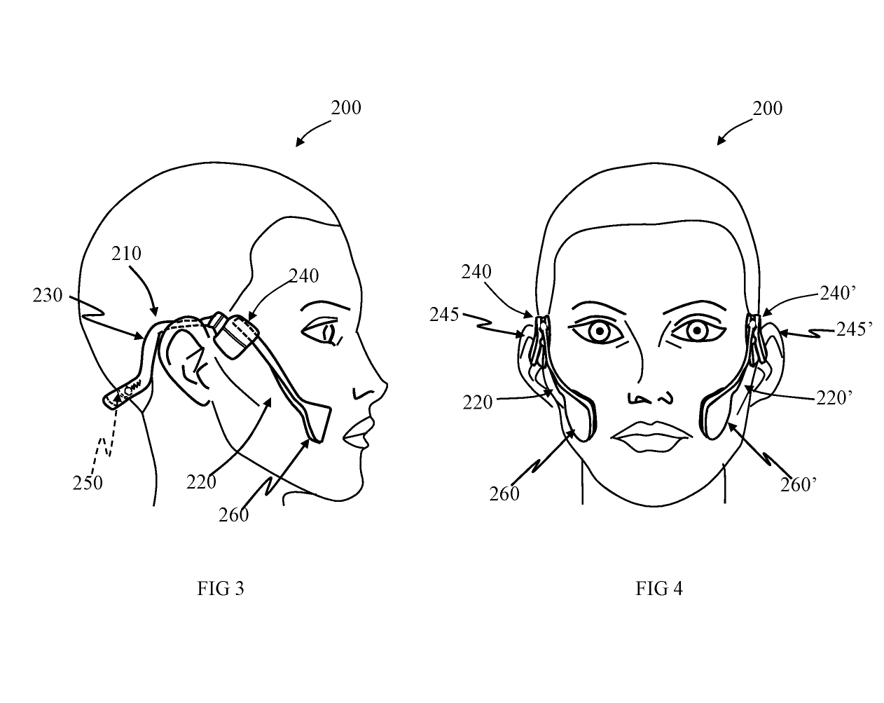

FIG. 4 demonstrates a front view of the head 200 with the head attachment shown in FIG. 3. Right temporal element 240 is shown at the right side of the head in front of right ear and left temporal element 240′ of equivalent construction is shown at the left side of the head in front of the left ear. Right retainer element 220 is attached to the temporal element 240 and left retaining element 220′ is attached to the temporal element 240′. Supporting elements 260 and 260′ are positioned to restrict a relative to head movement of the face/cheek soft tissue mass which is particularly undesirable with a vigorous head movement.

Retainer elements can be a part of the head attachment structure, i.e. to be non-detachable. Alternatively, retainer elements (220, 220′) can be adjustable for a separation distance between corresponding supporting element (260, 260′) and temporal element (240, 240′). This can be accomplished by bending and/or stretching a corresponding retainer element to increase or reduce a separation distance, for instance, or each retaining element may include a telescopic construction to control its length.

In general, a device according to the present invention is in contact with head surface at a minimum five areas—at about the nape area of the head, right and left temporal areas, and right and left lower cheeks areas. The device according to the present invention is shaped to leave the chin free for movement thus not impairing the ability of a wearer to talk or eat. There is not a nose contact either to maintain an inconspicuous appearance. Portions of head attachment are located between the helix of ears 245 and 245′ and corresponding temporal regions of the head above the ears and can be shaped and sized similar to a common shape and size of eyeglasses or sunglasses arms. In this case these portions of the head attachment body are also in contact with the head though a head attachment body can be shaped to be exteriorly of the ears without contacting the head at the temporal area except by the temporal elements.

FIG. 5 demonstrates a close up of the side view of the device according to the present invention shown in FIG. 3. Head attachment 210 is attached to the head at its front side by temporal element 240, where then the head attachment 210 is attached on its other side to the front end of the flexible solid head attachment body 230. Another temporal element is similarly connected to the other side of the head adjustment body 230, and also by the elastic band 250 that supports the head attachment body 230 at about the nape of the head. The elastic band 250 can be a solid adjustable part of the head adjustment body 230 through the use of a screw or gear mechanism. In another embodiment the head attachment body 230 is supported by the elastic band 250 at about the nape of the head. A separation distance between the nape of the head and temporal element 240 is adjustable by the nub 280 to allow for head attachment 210 fixation onto the head. The elastic band 250 adjustment can be by different means, in another embodiment, notches 290 are included at the head attachment body 230 to fix the nub position at different locations along the head attachment body 230 thus adjusting for a separation distance between the nape of the head and temporal elements.

Retainer element can be of different shapes. In another embodiment, the right retaining element 220 consists of supporting element 260 and retainer arm 330. Retainer arm 330 can be made of flexible material (such as spring metal, polymers, composites and/or combinations thereof) to enable adjustment of a separation distance between the supporting element 260 and the temporal element 240. Another option is to control a portion 335 of the retainer arm 330 that is within the temporal element 240 to control a separation distance between supporting element 260 and temporal element 240. There could be also a disposable pad 300 attached to the internal area of the supporting element 260. As previously discussed herein, the left retaining element is a mirror symmetry to the right retaining element 220.

There could be an adjustment element 270 as part of the front end of the attachment body 230 which is connected with the temporal element 240. Adjustment element 270 is to adjust an angle between the temporal element 240 and temporal surface of the head. Simultaneously, the adjustment element 270 could also adjust for a position of the supporting element 260 in respect to the corresponding cheek surface. This allows for addition adjustment for cheek shape and tension on the cheek by the supporting element 260 of the retainer element 220. Equivalently, an adjustment element can also be included at the left side of the head.

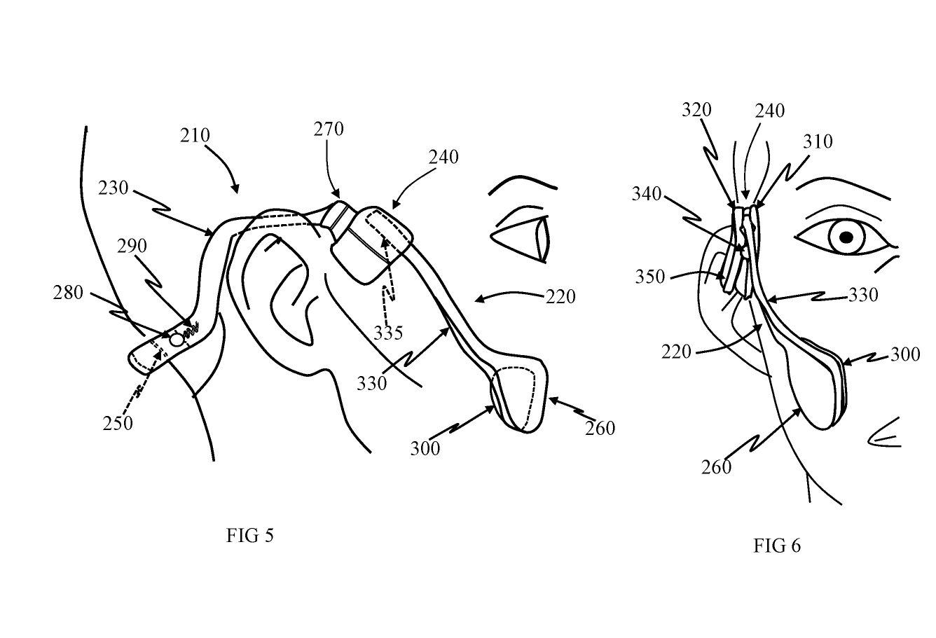

FIG. 6 demonstrates a front view of the head with right half of the head attachment that includes right retainer element 220 shown in FIG. 5. Retainer element 220 includes supporting element 260 for cheek support with a disposable pad 300 being in contact with the cheek itself. The supporting element 260 is connected to the temporal element 240 via the retainer arm 330.

In another embodiment, the temporal element 240 is constructed as a spring clamp structure to fixate the retainer element 220 to it. The spring clamp temporal element 240 consists of fixed internal jaw 310 and movable (pivotable) external jaw 320 with the retainer arm 330 fixed in between. A separation between the upper parts of fixed internal jaw 310 and movable external jaw 320 is increased by pressing the lower part 350 of the external jaw 320 for the external jaw 320 rotation around pivot 340. A spring or bias creates a force such that the upper portion of movable external jaw 320 is biased against the fixed internal jaw 310 thereby capturing retainer arm therebetween. Upon the movable external jaws 320 release, the spring closes the upper parts of the jaws 320 and 310 over the retainer arm 330 thereby fixing the retainer element's 220 position for a desired separation distance between the supporting element 260 and temporal element 240. There is a separation space between fixed jaw 310 and movable jaw 320 even with the help of the retainer element 220 in between. This space is to accept eyeglasses or sunglasses arms to allow its common position during eyeglasses or sunglasses wear.

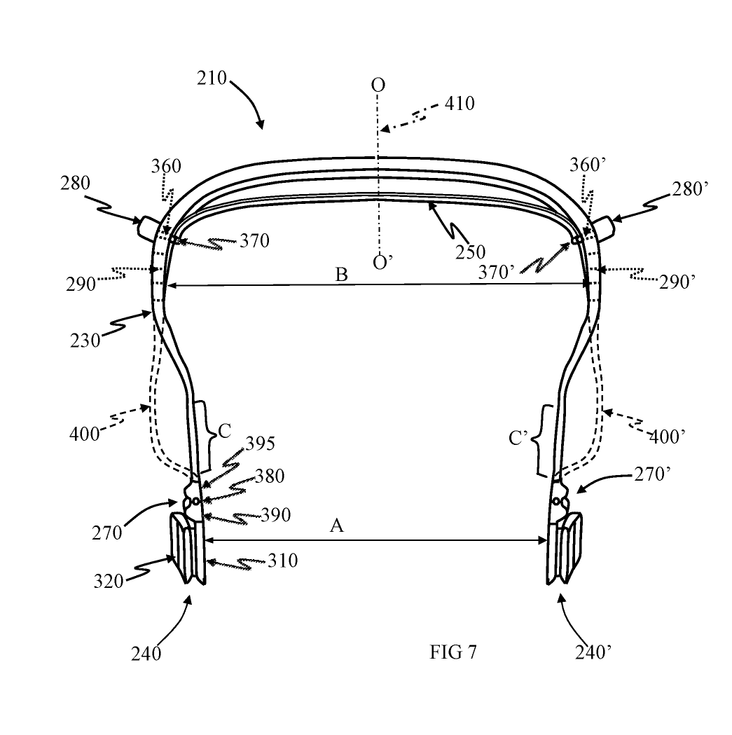

FIG. 7 demonstrates a top view of the head attachment 210 as taken from FIG. 4 and without the head, where it manifests a largely symmetrical structure about axis 410 from point O to point O′. Temporal elements 240 and 240′ are located at front ends of the head attachment body 230. Temporal elements can be constructed as spring clamp with fixed jaw 310 and movable jaw 320 but it could be other designs for retainer elements fixation to a head attachment of the device according to the present invention. The head attachment 210 includes flexible head attachment body 230 made of an elastic material (metal, plastic or their combination) in order to vary distance A for fitting the head attachment body 230 to a variety of head sizes.

Head attachment body 230 may include an adjustable element 270 with temporal element 240 connected to it, i.e. connected to the internal jaw 310. Correspondingly, adjustable element 270′ at the other end of the head adjustment body 230 is connected to the temporal element 240′. Both adjustable elements 270 and 270′ are shown to be constructed similar and therefore are explained from the example of the right adjustable element 270. The adjustable element 270 consists of fixed element 395 and movable element 390 which can be rotated/pivoted around axis 380 to fix the movable element 390 at different angle from the fixed element 395, which is done by a gear mechanism at and/or along the axis 380.

The head attachment body 230 includes section C at one side and largely mirror symmetrical section C′ at the other side, where each section is constructed to be placed between the corresponding helix of ear and temporal area of the head in the position when the head attachment 210 is attached to the head. The other option to have the head attachment body shaped to be outside each ear as shown by sections 400 and 400′. The back section of the head attachment 210 is designed to be oversized of distance B in width and length to avoid a contact of the back section at the back of the head. This is to minimize interference with a hair style, head shape and to allow for easier elastic band 250 adjustment.

The adjustable elastic band 250 is located internally to the back section of the head adjustment body 230. It is designed to be in contact at about the nape of the head as part of head attachment 210 fixation onto the head. The elastic band 250 is held by the edge elements 370 and 370′ at the internal surface of the head adjustment body 230. The edge element 370 is attached to the nub 280 via the connecting element 360. Similarly, edge element 370′ is connected to the nub 280′ via connecting element 360′. Each nub 280 or 280′ can be moved by hand along the head attachment body 230 to change a separation distance between the nape of the head and each temporal element 240 and 240′ and then fixing elastic bend 250 position by fixed the locations of corresponding connecting element 360 and/or 360′ within the notches 290 or 290′. As a result, the head attachment 210 is fixed onto the head of a verity of shapes and sizes with the help of the elastic band 250 and temporal elements 240 and 240′.

What is claimed is:

1. A device to be placed onto and/or around a person's head and neck area configured to reduce tension in facial ligaments, the device comprising:
a generally U-shaped head attachment body defining a left side connected to a right side, the head attachment body configured to be disposed behind the person's head and neck area and to be in contact at or near a nape area of the person, where the left side of the head attachment body comprises a left attachment portion and the right side of the head attachment body comprises a right attachment portion;
a left temporal element attached to the left attachment portion of the head attachment body, the left temporal element configured to be in contact with the person's left temporal area;
a right temporal element attached to the right attachment portion of the head attachment body, the right temporal element configured to be in contact with the person's right temporal area;
a left cheek supporting element attached to either the left temporal element or the left attachment portion of the head attachment body, the left cheek supporting element configured to be in contact with the person's left cheek area; and
a right cheek supporting element attached to either the right temporal element or the right attachment portion of the head attachment body, the right cheek supporting element configured to be in contact with the person's right cheek area.

2. The device of claim 1, wherein the left temporal element comprises a left fixed internal jaw pivotably connected to a left movable external jaw about a left pivot, wherein the left movable external jaw is biased into a left closed position towards the left fixed internal jaw by a left spring or bias element.

3. The device of claim 2, including a left retaining arm extending from the left cheek supporting element, wherein an end of the left retaining arm is captured between the left fixed internal jaw and the left movable external jaw in the left closed position.

4. The device of claim 3, including a left separation space between the left fixed internal jaw and the left movable external jaw while capturing the left retaining arm, wherein the left separation space is configured to accept an auxiliary pair of glasses or sunglasses for the person to wear.

5. The device of claim 4, wherein the right temporal element comprises a right fixed internal jaw pivotably connected to a right movable external jaw about a right pivot, wherein the right movable external jaw is biased into a right closed position towards the right fixed internal jaw by a right spring or bias element.

6. The device of claim 5, including a right retaining arm extending from the right cheek supporting element, wherein an end of the right retaining arm is captured between the right fixed internal jaw and the right movable external jaw in the right closed position.

7. The device of claim 6, including a right separation space between the right fixed internal jaw and the right movable external jaw while capturing the right retaining arm, wherein the right separation space is configured to accept an auxiliary pair of glasses or sunglasses for the person to wear.

8. The device of claim 1, including a left adjustment element connecting to and between the left attachment portion and the left temporal element, the left adjustment element comprising a left fixed element pivotably connected to a left movable element, where the left fixed element is attached to the left attachment portion and the left movable element is connected to the left temporal element, wherein the left movable element pivots about a left axis in relation to the left fixed element, wherein the person can adjust and lock a left angle between the left fixed element and the left movable element thereby allowing adjustment of the left temporal element in contact with the person's left temporal area.

9. The device of claim 8, including a right adjustment element connecting to and between the right attachment portion and the right temporal element, the right adjustment element comprising a right fixed element pivotably connected to a right movable element, where the right fixed element is attached to the right attachment portion and the right movable element is connected to the lef right t temporal element, wherein the right movable element pivots about a right axis in relation to the right fixed element, wherein the person can adjust and lock a right angle between the right fixed element and the right movable element thereby allowing adjustment of the right temporal element in contact with the person's right temporal area.

10. The device of claim 1, wherein the left and right attachment portions are configured to reside between the person's ear and the person's head when the device is placed onto and/or around the person's head.

11. The device of claim 1, wherein the left and right attachment portions are configured to reside outside the person's ear and the person's head when the device is placed onto and/or around the person's head.

12. The device of claim 1, wherein a left separation distance between the left cheek supporting element and the left temporal element is adjustable.

13. The device of claim 12, wherein a right separation distance between the right cheek supporting element and the right temporal element is adjustable.

14. The device of claim 1, wherein the generally U-shaped head attachment body defining a left side connected to a right side comprises a solid but resiliently flexible material.

15. The device of claim 1, including an elastic band disposed on an inside and/or interior of the head attachment body, where one end of the elastic band is movably connected along the left side of the head attachment body and where the other end of the elastic band is movably connected along the right side of the head attachment body.

16. A device to be placed onto and/or around a person's head and neck area configured to reduce tension in facial ligaments, the device comprising:
a generally U-shaped head attachment body defining a left side connected to a right side, the head attachment body configured to be disposed behind the person's head and neck area at or near a nape area of the person, where the left side of the head attachment body comprises a left attachment portion and the right side of the head attachment body comprises a right attachment portion;
an elastic band disposed on an inside or interior of the head attachment body to be in contact at or near a nape area of the person where one end of the elastic band is movably connected along the left side of the head attachment body and where the other end of the elastic band is movably connected along the right side of the head attachment body;
a left temporal element attached to the left attachment portion of the head attachment body, the left temporal element configured to be in contact with the person's left temporal area, wherein the left temporal element comprises a left fixed internal jaw pivotably connected to a left movable external jaw about a left pivot, wherein the left movable external jaw is biased into a left closed position towards the left fixed internal jaw by a left spring or bias element;
a right temporal element attached to the right attachment portion of the head attachment body, the right temporal element configured to be in contact with the person's right temporal area, wherein the right temporal element comprises a right fixed internal jaw pivotably connected to a right movable external jaw about a right pivot, wherein the right movable external jaw is biased into a right closed position towards the right fixed internal jaw by a right spring or bias element;
a left cheek supporting element attached to either the left temporal element or the left attachment portion of the head attachment body, the left cheek supporting element configured to be in contact with the person's left cheek area;
a left retaining arm extending from the left cheek supporting element, wherein an end of the left retaining arm is captured between the left fixed internal jaw and the left movable external jaw in the left closed position;
a right cheek supporting element attached to either the right temporal element or the right attachment portion of the head attachment body, the right cheek supporting element configured to be in contact with the person's right cheek area; and
a right retaining arm extending from the right cheek supporting element, wherein an end of the right retaining arm is captured between the right fixed internal jaw and the right movable external jaw in the right closed position.

17. The device of claim 16, including a left adjustment element connecting to and between the left attachment portion and the left temporal element, the left adjustment element comprising a left fixed element pivotably connected to a left movable element, where the left fixed element is attached to the left attachment portion and the left movable element is connected to the left temporal element, wherein the left movable element pivots about a left axis in relation to the left fixed element, wherein the person can adjust and lock a left angle between the left fixed element and the left movable element thereby allowing adjustment of the left temporal element in contact with the person's left temporal area.

18. The device of claim 17, including a right adjustment element connecting to and between the right attachment portion and the right temporal element, the right adjustment element comprising a right fixed element pivotably connected to a right movable element, where the right fixed element is attached to the right attachment portion and the right movable element is connected to the left right t temporal element, wherein the right movable element pivots about a right axis in relation to the right fixed element, wherein the person can adjust and lock a right angle between the right fixed element and the right movable element thereby allowing adjustment of the right temporal element in contact with the person's right temporal area.

19. The device of claim 18, wherein a left separation distance between the left cheek supporting element and the left temporal element is adjustable.

20. The device of claim 19, wherein a right separation distance between the right cheek supporting element and the right temporal element is adjustable.
